(19) United States
(12) Patent Application Publication
(10) Pub. No.: US 2026/0212657 A1
(43) Pub. Date: Jul. 23, 2026

(54) MULTIMODAL FEDERATED LEARNING METHOD, SYSTEM AND DEVICE BASED ON MEDICAL LARGE MODEL

(71) Applicants: Peking Union Medical College, Beijing (CN); University of Science and Technology Beijing, Beijing (CN)

(72) Inventors: Meihua PIAO, Beijing (CN); Hongzhen CUI, Beijing (CN); Yunfeng PENG, Beijing (CN); Haoming MA, Beijing (CN); Sijia LI, Beijing (CN); Xiaoyue ZHU, Beijing (CN); Shenhui NING, Beijing (CN); Xingyi TANG, Beijing (CN); Li QIAO, Beijing (CN)

(21) Appl. No.: 19/082,862

(22) Filed: Mar. 18, 2025

(30) Foreign Application Priority Data
Jan. 17, 2025 (CN) 202510081093.3

(51) Int. Cl. G06V 10/80; G06F 16/353; G06V 10/762

(52) U.S. Cl. CPC G06V 10/803; G06F 16/353; G06V 10/762

(57) ABSTRACT

Provided are a multimodal federated learning method, system and device based on a medical large model. The method includes: performing local training on a received initial global model on a medical institution client to obtain a local model, and uploading the local model to a server; performing cross-validation, implementing global training by evaluating and retaining high-quality model features; iterating until the optimized global model meets a first preset condition whereupon the learning is stopped; clustering the medical institution clients to obtain a plurality of clusters, inputting the optimal local model into the corresponding cluster to undergo intra-cluster aggregation to obtain a cluster model; feeding the cluster model into each medical institution client to undergo local training until a second preset condition is met, and then uploading a secondary optimized local model of each medical institution client to the server to undergo aggregation to obtain an optimal federated global model.

### CROSS REFERENCE TO RELATED APPLICATION

This patent application claims the benefit and priority of Chinese Patent Application No. 2025100810933, filed with the China National Intellectual Property Administration on Jan. 17, 2025, the disclosure of which is incorporated by reference herein in its entirety as part of the present application.

### TECHNICAL FIELD

The present disclosure relates to the field of federated learning, and in particular, to a multimodal federated learning method, system and device based on a medical large model.

### BACKGROUND

With the rapid development of artificial intelligence and machine learning technologies, data-driven applications of medical data are increasing. Large quantities of medical data can be analyzed to train models for wide use in disease prediction, diagnosis support, treatment decision optimization, and the like. The effect of model training is closely related to the scale of the dataset. However, medical data is highly related to patient privacy and sensitivity. Conventional integrated machine learning methods need to store and process datasets in a centralized manner, thereby posing a problem of data privacy leakage and a security problem. The single-modal data used in the conventional federated learning can provide only limited information, thereby resulting in incompleteness or loss of information. In addition, the conventional federated learning integrates the local model parameter features together without considering the quality of local model features, thereby resulting in limited model performance.

### SUMMARY

An objective of the present disclosure is to provide a multimodal federated learning method, system and device based on a medical large model to ensure accuracy and efficiency of global model training while enhancing generalizability of the model.

To achieve the above objective, the present disclosure provides the following technical solution:

According to a first aspect, the present disclosure provides a multimodal federated learning method based on medical large model, including:
sending, by a server, an initial global model to each medical institution client, where the initial global model is a medical large model;
performing local training on the received initial global model on any one of the medical institution clients by use of a multimodal feature fusion dataset to obtain a local model, and uploading the local model to the server;
performing cross-validation on a plurality of local models on the server, and implementing global training by evaluating and retaining high-quality model features to obtain an optimized global model;
updating the initial global model to the optimized global model when the optimized global model fails to meet a first preset condition, and then returning to the step of sending the initial global model to each medical institution client through the server, so as to perform a next federated learning operation; iterating until the optimized global model meets the first preset condition whereupon the learning is stopped, marking the optimized global model as an optimal global model, and marking a local model corresponding to each medical institution client as an optimal local model;
clustering the medical institution clients based on the optimal global model to obtain a plurality of clusters, and then inputting the optimal local model of each medical institution client into the corresponding cluster to undergo intra-cluster aggregation to obtain a cluster model;
feeding the cluster model of each cluster into each corresponding medical institution client to undergo local training until a second preset condition is met, whereupon a secondary optimized local model is obtained; and
uploading the secondary optimized local model of each medical institution client to the server to undergo aggregation to obtain an optimal federated global model.

According to a second aspect, the present disclosure provides a multimodal federated learning system based on a medical large model, including:
a sending module, configured to send an initial global model to each medical institution client through a server, where the initial global model is a medical large model;
a local multi-round training module, configured to perform local training on the received initial global model on any one of the medical institution clients by use of a multimodal feature fusion dataset to obtain a local model, and upload the local model to the server;
a cross-validation optimum module, configured to perform cross-validation on a plurality of local models on the server, and implement global training by evaluating and retaining high-quality model features to obtain an optimized global model;
a cyclic learning module, configured to: update the initial global model to the optimized global model when the optimized global model fails to meet a first preset condition, and then return to the step of sending the initial global model to each medical institution client through the server, so as to perform a next federated learning operation; repeat this process until the optimized global model meets the first preset condition whereupon the learning is stopped, mark the optimized global model as an optimal global model, and mark a local model corresponding to each medical institution client as an optimal local model;
a clustering module, configured to cluster the medical institution clients based on the optimal global model to obtain a plurality of clusters, and then input the optimal local model of each medical institution client into the corresponding cluster to undergo intra-cluster aggregation to obtain a cluster model, where
the sending module is further configured to: feed the cluster model of each cluster into each corresponding medical institution client to undergo local training in the local multi-round training module until a second preset condition is met, whereupon a secondary optimized local model is obtained; and
an ultimate model determining module, configured to: aggregate the secondary optimized local model of each medical institution client uploaded to the server, so as to obtain an optimal federated global model.

According to a third aspect, the present disclosure provides a computer device, including: a memory, a processor, and a computer program stored in the memory and executable on the processor. The processor executes the computer program to implement the multimodal federated learning method based on a medical large model.

According to the specific embodiments provided herein, the present disclosure achieves at least the following technical effects: the present disclosure provides a multimodal federated learning method, system and device based on a medical large model, performs local model training by using a multimodal feature fusion dataset, performs cross-validation on a plurality of local models on the server, and performs global training by evaluating and retaining high-quality model features, thereby providing a more comprehensive data perspective while ensuring the accuracy and efficiency of the global model training, enhancing the accuracy and generalizability of the model, and being applicable in complex healthcare scenarios of different diseases. After the optimal global model is obtained, the medical institution clients are clustered to obtain a plurality of clusters, and then intra-cluster aggregation is performed to obtain a cluster model. This arrangement considers the characteristics of endemic diseases, and the medical institution clients of high consistency are clustered, thereby implementing fine-grained characterization of the images of different categories of medical institution clients. Finally, the present disclosure fuses multimodal data, breaks down data silos in medical institutions through distributed database training, and protects user privacy securely.

### BRIEF DESCRIPTION OF THE DRAWINGS

To describe the technical solutions in some embodiments of the present disclosure or the prior art more clearly, the following outlines the drawings to be used in the embodiments. Evidently, the drawings outlined below are merely a part of embodiments of the present disclosure. A person of ordinary skill in the art may derive other drawings from the outlined drawings without making any creative efforts.

FIG. 1 is a schematic flowchart of a multimodal federated learning method based on a medical large model according to an embodiment of the present disclosure;

FIG. 2 is a schematic diagram of multimodal data fusion of medical institution clients according to the present disclosure;

FIG. 3 is a schematic flowchart of a federated learning based on graph clustering according to the present disclosure;

FIG. 4 is a schematic clustering diagram of a feature enhancement graph of medical institution clients according to the present disclosure; and

FIG. 5 is a schematic structural diagram of a computer device according to an embodiment of the present disclosure.

### DETAILED DESCRIPTION OF THE EMBODIMENTS

The following describes the technical solutions in some embodiments of the present disclosure clearly in detail with reference to the drawings appended hereto. Evidently, the described embodiments are merely a part of but not all of the embodiments of the present disclosure. All other embodiments derived by a person of ordinary skill in the art based on the embodiments of the present disclosure without making any creative effort fall within the protection scope of the present disclosure.

To make the objectives, features, and advantages of the present disclosure more apparent and comprehensible, the following describes the present disclosure in more detail with reference to accompanying drawings and specific embodiments.

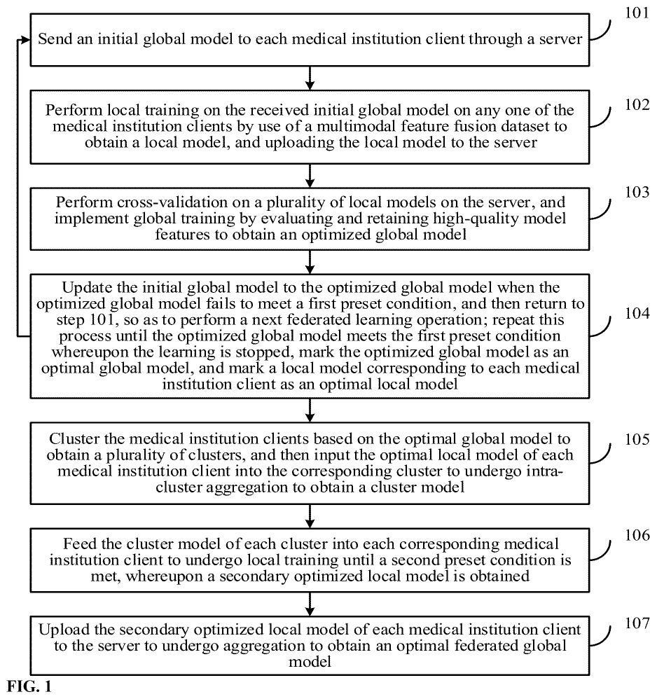

In an exemplary embodiment, as shown in FIG. 1, a multimodal federated learning method based on a medical large model is provided. The method is executed by a computer device, and specifically, may be executed by a computer device such as a terminal or a server alone, or may be executed by a terminal or server together. In an embodiment of the present disclosure, the method includes the following steps 101 to 107.

Step 101: A server sends an initial global model to each medical institution client. Specifically, the server transmits a medical global model into a preprocessing module, randomly initializes all model parameters (or features) in the preprocessing module to obtain an initialized global model, ƒ¹ and then transmits the initialized global model into the medical institution client as a local initialized model.

Step 102: Perform local training on the received initial global model on any one of the medical institution clients by use of a multimodal feature fusion dataset to obtain a local model, and upload the local model to the server.

In an application example, as shown in FIG. 2, the process of acquiring the multimodal feature fusion dataset includes: transferring the multimodal dataset of each corresponding medical institution collected by N medical institution clients

$$\{ {D}_{1}^{0} , {D}_{2}^{0} , … , {D}_{i}^{0} , … , {D}_{N}^{0} \}$$

into a multimodal feature fusion module, and executing the following steps (21) to (23) to implement feature fusion and obtain local enhanced feature data. The local data of the medical institution with enhanced features is a multimodal feature fusion dataset {D₁, D₂, . . . , Dᵢ, . . . , D_{N}}.
(21) Acquire text data, voice data, and image data from the medical institution server;
(22) Extract features of the text data, voice data, and image data of the medical institution server separately to obtain text features, voice features, and image features; and
(23) Perform, based on an attention mechanism, multimodal feature fusion on the text features, the voice features, and the image features to obtain a multimodal feature fusion dataset. Specifically, the correlation and difference of features between different modalities are explored by an attention mechanism, and an attention weight is added to each different modality. The input of the attention mechanism is formed of query (Q), a key (K), and a value (V). The calculation formula of the attention weight is as follows:

$${β}_{i} = \frac{exp ( s ( {x}_{i} , q ) )}{{\sum }_{o = 1}^{N} exp ( s ( {x}_{o} , q ) )} .$$

In the formula above, s(xᵢ,q) is an attention mechanism probability output, expressed by the following formula: s(xᵢ, q)=V^{T} tanh (Wxᵢ+U_{q}); and V, W, and U serve as a to-be-learned reference matrix, and is applied to the input feature after the attention weight is obtained.

In another application example, in a process of performing local training on the received initial global model by use of a multimodal feature fusion dataset, the following loss function is used:

$${L}_{i} ( {f}_{i}^{p} ) = \frac{1}{{D}_{i}} {\sum }_{k ∈ {D}_{i}} {F}_{k} ( {f}_{i}^{p} ) .$$

In the formula above,

$${L}_{i} ( {f}_{i}^{p} )$$

is a value of the loss function of

$${f}_{i}^{p} , {f}_{i}^{p}$$

is a model feature of a medical institution client i in the p^{th} round, Dᵢ is multimodal feature fusion dataset of the medical institution client i, k is any one multimodal feature fusion datum in Dᵢ, and a purpose of

$${F}_{k} ( {f}_{i}^{p} )$$

is to find an optimal parameter ƒ* of the model to minimize the loss function; and ƒ* is formalized as:

$${f}^{*} = arg min {L}_{i} ( {f}_{i}^{p} ) .$$

$$F ( {f}_{i}^{p} )$$

is updated iteratively for several times by a gradient descent method to minimize a local loss by using the following formula:

$${f}_{i}^{p} = {f}_{i}^{p - 1} - λ ∇ F ( {f}_{i}^{p} ) .$$

In the formula above, λ is a step length, and λ>0;

$${f}_{i}^{p - 1}$$

is a model feature of the medical institution client i in the (p−1)^{th} round, and

$$∇ F ( {f}_{i}^{p} )$$

is a gradient operator. After t iterations, each medical institution client obtains an updated local model

$${f}_{i}^{p} ,$$

and then inputs the model into a cross-validation optimal module on the server.

In another application example, the initial global model is a medical large model. In other words, the present disclosure uses a medical large model as a federated learning distributed database training method. The medical large model is used for implementing image segmentation, disease knowledge reasoning, or clinical auxiliary diagnosis. Based on the above different functional needs, a model output corresponding to the model input is set. For example, when image segmentation is required, the model input of the medical large model is multimodal feature fusion data, and the model output of the medical large model is a segmentation result of the image data in the multimodal feature fusion data. For example, when disease knowledge reasoning is required, the model input of the medical large model is multimodal feature fusion data, and the model output of the medical large model is the type of the inferred disease or the stage in which the inferred disease is located (for example, negative, positive, or the like).

Step 103: Perform cross-validation on a plurality of local models on the server, and implement global training by evaluating and retaining high-quality model features to obtain an optimized global model. The global training is to aggregate the local models of all medical institution clients. A conventional aggregation method includes hard voting and soft voting. In an embodiment of the present disclosure, by evaluating the quality of the model feature of the local model of each medical institution client, high-quality features are screened and retained, and low-quality features are rejected. The method for evaluating the quality of model features includes a filter method, a wrapper method, and an embedded method. This embodiment uses the filter method as an example to give a detailed description. The method for evaluating the quality of model features includes the following steps:

First, the server establishes a model by using the features of the local model of each medical institution client, and performs cross-validation to obtain a score of the corresponding model feature. The server selects q model features with the highest scores, q≤N, to form a high-quality feature set. The rest features constitute a low-quality feature set. The global model in the (p+1)^{th} round is as follows:

$${f}^{p + 1} = \frac{{\sum }_{i = 1}^{N} ω \vert {D}_{i} \vert {f}_{i}^{p}}{{\sum }_{i = 1}^{N} \vert {D}_{i} \vert} .$$

In the formula above, ƒ^{p+1} is the optimized global model in the p+1^{th} round, N is the number of medical institution clients; and ω is a model feature weight. When the feature is of the highest quality, the value of ω is 1; otherwise, the value is 0.

$${f}_{i}^{P}$$

is a model feature of the medical institution client i in the pth round. Dᵢ is the multimodal feature fusion dataset of the medical institution client i.

Step 104: Update the initial global model to the optimized global model when the optimized global model fails to meet a first preset condition, and then return to the step of sending the initial global model to each medical institution client through the server, so as to perform a next federated learning operation; repeat this process until the optimized global model meets the first preset condition whereupon the learning is stopped, mark the optimized global model as an optimal global model, and mark a local model corresponding to each medical institution client as an optimal local model.

In an application example, the p+1^{th}-round optimized global model ƒ^{p+1} obtained in step 103 is sent to each medical institution client through step 101, and local training is performed in the local multi-round training module to obtain an updated local model

$${f}_{i}^{p + 2} ,$$

and then this model

$${f}_{i}^{p + 2}$$

is uploaded to the cross-validation optimum module of the server to obtain the p+2^{th} round global model ƒ^{p+2}. The above steps are repeated until the optimized global model reaches the optimal threshold, so that the optimal global model ƒ^{p} is obtained, and the optimal local model

$${f}_{i}^{P}$$

of each medical institution client is obtained.

Step 104: Cluster the medical institution clients based on the optimal global model ƒ^{p} so that the medical institution clients of the same feature are aggregated into one cluster to obtain a plurality of clusters; and then input the optimal local model of each medical institution client into the corresponding cluster to undergo intra-cluster aggregation to obtain a cluster model, that is, an intra-cluster adaptive model.

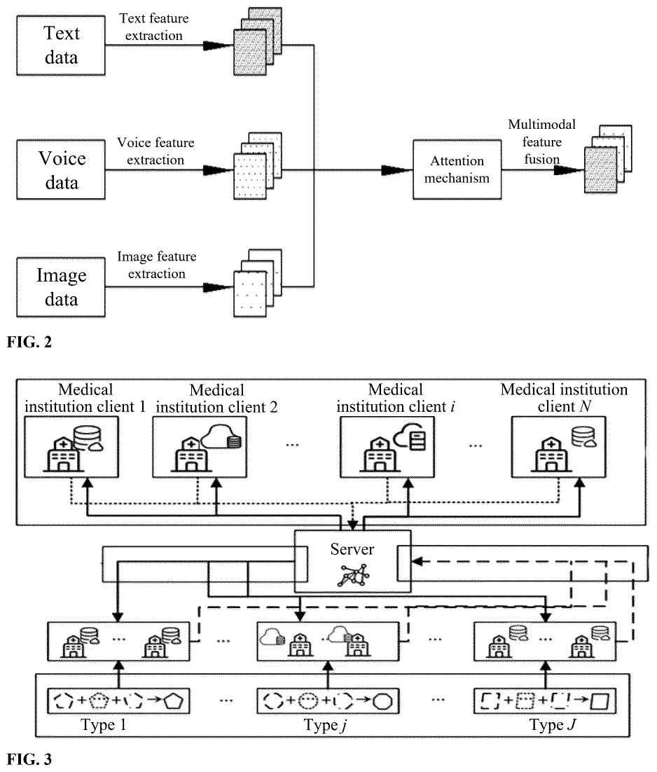

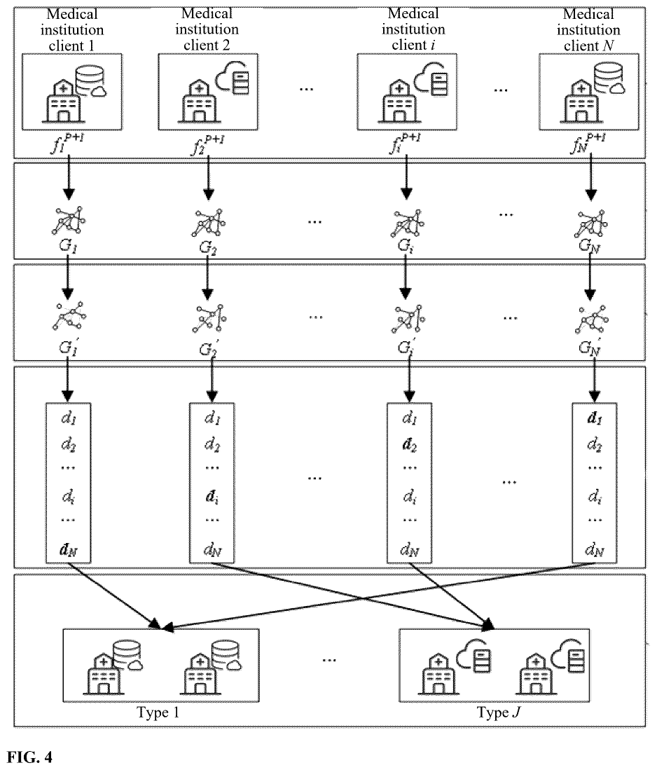

In another application example, as shown in FIG. 3 and FIG. 4, the clustering the medical institution clients based on the optimal global model to obtain a plurality of clusters includes:
Step (41): The server sends the optimal global model to each medical institution client, performs local training to obtain an optimized local model, and uploads the optimized local model to the server.
Step (42): Perform feature mapping on the plurality of the optimized local models by using a multi-layer neural network, so as to obtain a feature mapping graph Gᵢ, where the model feature of each optimized local model is converted into a weight of each edge in the corresponding feature mapping graph.
Step (43): Perform personalized enhanced feature extraction on the feature mapping graph to obtain a feature enhancement graph G′ᵢ by using the following function:

$${H}_{t}^{l} = \{ \begin{matrix}1 , \vert ω ( {e}_{t}^{l} ) - {ω ( {e}_{t}^{l} )}_{i} \vert \geq δ , {e}_{t}^{l} ∈ {E}_{l}^{P} , {E}_{l , i}^{P + 1} \\ 0 , \vert ω ( {e}_{t}^{l} ) - {ω ( {e}_{t}^{l} )}_{i} \vert < δ , {e}_{t}^{l} ∈ {E}_{l}^{P} , {E}_{l , i}^{P + 1}\end{matrix} .$$

In the function above, when a value of

$${H}_{t}^{l}$$

is 1, the value means that the feature weight is valid before and after the feature mapping graph is enhanced, and an edge

$${e}_{t}^{l}$$

is retained; when the value of

$${H}_{t}^{l}$$

is 0, the value means that the feature weight is invalid before and after the feature mapping graph is enhanced, and the edge

$${e}_{t}^{l}$$

is deleted;

$$ω ( {e}_{t}^{l} ) and {ω ( {e}_{t}^{l} )}_{i}$$

are weights of the edge

$${e}_{t}^{l}$$

before and after the feature mapping graph is enhanced, respectively; δ is a threshold; and

$${E}_{l}^{P} and {E}_{l , i}^{P + 4}$$

are a set of edges connecting an (s−1)^{th} layer and an s^{th} layer before and after the feature mapping graph is enhanced, respectively, satisfying: 1≤s≤S.

Step (44): Convert the feature enhancement graph into a similarity distance vector, for example but without being limited to, perform the following steps on the server:
1) Generate a weighted adjacency matrix Wᵢ based on the feature enhancement graph.
2) Obtain a Laplacian operator Lᵢ=Oᵢ−Wᵢ of the feature enhancement graph based on an adjoint diagonal matrix Oᵢ of the weighted adjacency matrix Wᵢ.
3) Calculate an eigenvalue sequence λᵢ and an eigenvector sequence of the Laplacian operator of the feature enhancement graph. Perform calculation by using the following formula: Lᵢivᵢ=λᵢvᵢ, where vᵢ is an eigenvector sequence of Lᵢ, and specifically, vᵢ={vᵢ₁, vᵢ₂, . . . , vᵢₙ}, and λᵢ={λᵢ₁, λᵢ₂, . . . , λᵢₙ}.
4) Calculate eigenvalue similarity based on the eigenvalue sequence by using the following formula:

$$D i {s}_{eig} ( i , j ) = \sqrt{{\sum }_{k = 1}^{n} {( {λ}_{ik} - {λ}_{j , jk} )}^{2}} .$$

5) Calculate eigenvector similarity, and specifically, the F-norm between the feature vector sequences of the medical institution client, based on the eigenvector sequence by using the following formula:

$$D i {s}_{v e c} ( i , j ) = \sqrt{{\sum }_{k = 1}^{n} {\sum }_{I = 1}^{n} {( {v}_{i , kI} - {v}_{j , kI} )}^{2}} .$$

6) Perform weighting by using the following formula, so as to calculate global similarity:

$${Dis}_{comb} ( i , j ) = α {Dis}_{eig} ( i , j ) + ( 1 - α ) {Dis}_{vec} ( i , j ) .$$

7) Store the global similarity of each feature enhancement graph as the similarity distance vector Disᵢ=[Disᵢ₁, Disᵢ₂, . . . , Disᵢⱼ, . . . , Dis_{iN}] of the corresponding medical institution client. In the similarity distance vector above, Disᵢⱼ is a distance between the feature enhancement graph

$${G}_{i}^{′}$$

and the feature enhancement graph

$${G}_{j}^{′} .$$

α∈[0,1] is a weight parameter, and depends on the actual training environment; Dis_{comb}(i, j) is the global similarity between the feature enhancement graph

$${G}_{i}^{′}$$

and the feature enhancement graph

$${G}_{j}^{′} ;$$

Dis_{eig}(i, j) is the eigenvalue similarity between the feature enhancement graph

$${G}_{i}^{′}$$

and the feature enhancement graph

$${G}_{j}^{′} ;$$

Dis_{vec}(i, j) is the eigenvector similarity between the feature enhancement graph

$${G}_{i}^{′}$$

and the feature enhancement graph

$${G}_{j}^{′} ;$$

λᵢₖ is the kth eigenvalue in the feature enhancement graph

$${G}_{i}^{′} ;$$

λⱼₖ is the kth eigenvalue in the feature enhancement graph

$${G}_{j}^{′} ;$$

v_{i,kl} is the kl^{th} eigenvector in the feature enhancement graph

$${G}_{i}^{′} ;$$

and v_{j,kl} is the kl^{th} eigenvector in the feature enhancement graph

$${G}_{j}^{′} .$$

Step (45): Cluster the plurality of medical institution clients by using a clustering algorithm based on the similarity distance vector, so as to obtain a plurality of clusters. Specifically, the clustering algorithm includes K-means clustering, hierarchical clustering, density-based clustering, and the like.

The above steps (41) to (45) may be implemented in the client clustering module of the server to obtain the 1^{st}-cluster medical institution client, the 2^{nd}-cluster medical institution client, and so on, the j^{th}-cluster medical institution client, and so on, the J^{th}-cluster medical institution client. In the present disclosure, a total of N medical institution clients are preset. After training, the N medical institution clients are aggregated into J clusters. The number of terminal devices in the j^{th} cluster is N_{j′}, j∈{1, 2, . . . , i, . . . , J},

$${\sum }_{j = 1}^{J} {N}_{j} = N .$$

After the medical institution clients are clustered, the server or the intra-cluster core medical institution client executes an intra-cluster aggregation module to perform intra-cluster aggregation on the updated local model of each medical institution in the cluster to obtain the (P+1)^{th}-round cluster model. The cluster model is:

$${f}_{j}^{P + 1} = \frac{{\sum }_{{n}_{j} = 1}^{{N}_{j}} \vert {D}_{{n}_{j}} \vert {f}_{{n}_{j}}^{P + 1}}{{\sum }_{{n}_{j} = 1}^{{N}_{j}} \vert {D}_{{n}_{j}} \vert} .$$

In the cluster model,

$${f}_{j}^{P + 1}$$

is the cluster model in a (P+1)^{th} round, Dₙ_{ⱼ} is the multimodal feature fusion dataset of a medical institution client nⱼ corresponding to a j^{th} cluster,

$${f}_{{n}_{j}}^{P + 1}$$

is the optimal local model of the medical institution client nⱼ corresponding to the j^{th} cluster in the (P+1)^{th} round, Nⱼ is a number of medical institution clients in the j^{th} cluster, satisfying:

$${\sum }_{j = 1}^{J} {N}_{j} = N ,$$

where N is the number of medical institution clients.

Step 106: Feed the cluster model of each cluster into each corresponding medical institution client to undergo local training until a second preset condition is met, whereupon a secondary optimized local model is obtained. Repeat the above operation of “inputting the optimal local model of each medical institution client into the corresponding cluster to undergo intra-cluster aggregation to obtain a cluster model; and feeding the cluster model of each cluster into each corresponding medical institution client to undergo local training” cyclically during this local training until the threshold is reached or the preset training time is met, whereupon the training is stopped.

Step 107: Upload the secondary optimized local model of each medical institution client to the server to undergo aggregation to obtain an optimal federated global model.

To sum up, considering the factor of endemic diseases, the present disclosure is described by using the graph clustering-based federated learning as an example. The graph clustering-based federated learning method described herein is suitable for incorporating medical institution clients into different clusters based on the features of endemic disease data, so as to further implement distributed database training. An exemplary application scenario of the method is: considering that the number of patients with primary hypertension in northern China is higher than that in southern China, the federated training clients are divided into a northern cluster and a southern cluster, thereby making the final results more practical and accurate.

Compared with the prior art, the present disclosure further exhibits the following advantages:
(1) The present disclosure provides a more comprehensive information perspective by fusing multi-modal medical data such as text, voice, and image, improves the accuracy and robustness of the federated training model of the medical large model, and can avoid the loss of information in unimodal data.
(2) The present disclosure can significantly improve the performance and generalizability of the global model by evaluating the quality of model features uploaded by the medical institution client and fusing only high-quality features to complete the global model iteration, so that the model of this method can achieve better results in complex healthcare scenarios of different diseases, paving the way for a new wave of artificial intelligence innovation in the healthcare industry.
(3) The present disclosure considers the characteristics of endemic diseases, and clusters highly consistent medical institution clients by a graph-clustering multimodal federated learning method, thereby implementing a fine-grained characterization of the profile of different categories of medical institutions.

Based on the same inventive concept, an embodiment of the present disclosure further provides a multimodal federated learning system based on a medical large model. The technical solution provided by this system to solve the problems is similar to the technical solution described in the above method. Therefore, the specific definitions in one or more embodiments of the system provided below may be learned with reference to the above definitions for the method, the details of which are omitted here.

The present disclosure discloses a multimodal federated learning system based on a medical large model, including: a sending module, a local multi-round training module, a cross-validation optimum module, a cyclic learning module, a clustering module, and an ultimate model determining module. The sending module, the cross-validation optimum module, the cyclic learning module, the clustering module, and the ultimate model determining module are disposed on the server. The local multi-round training module is disposed on the medical institution client.

The sending module is configured to send an initial global model to each medical institution client through a server.

The local multi-round training module, configured to perform local training on the received initial global model on any one of the medical institution clients by use of a multimodal feature fusion dataset to obtain a local model, and upload the local model to the server.

The cross-validation optimum module, configured to perform cross-validation on a plurality of local models on the server, and implement global training by evaluating and retaining high-quality model features to obtain an optimized global model.

The cyclic learning module is configured to update the initial global model to the optimized global model when the optimized global model fails to meet a first preset condition, and then return to the step of sending the initial global model to each medical institution client through the server, so as to perform a next federated learning operation; repeat this process until the optimized global model meets the first preset condition whereupon the learning is stopped, mark the optimized global model as an optimal global model, and mark a local model corresponding to each medical institution client as an optimal local model.

The clustering module is configured to: cluster the medical institution clients based on the optimal global model to obtain a plurality of clusters, and then input the optimal local model of each medical institution client into the corresponding cluster to undergo intra-cluster aggregation to obtain a cluster model.

The sending module is further configured to: feed the cluster model of each cluster into each corresponding medical institution client to undergo local training in the local multi-round training module until a second preset condition is met, whereupon a secondary optimized local model is obtained.

The ultimate model determining module is configured to: aggregate the secondary optimized local model of each medical institution client uploaded to the server, so as to obtain an optimal federated global model.

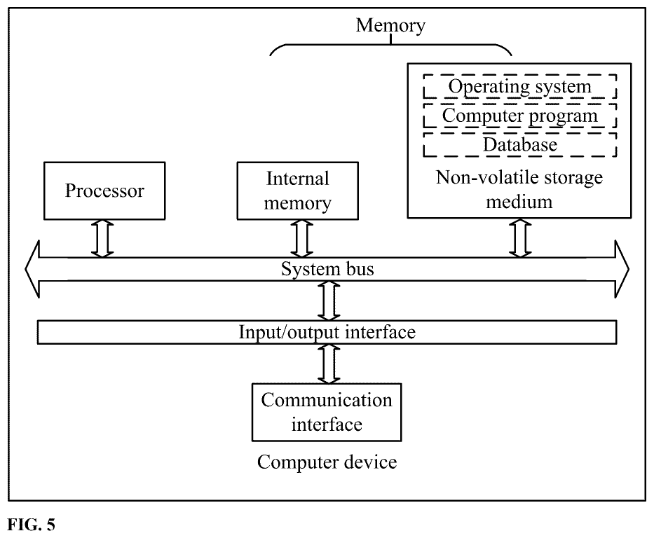

In an exemplary embodiment, a computer device is provided. The computer device may be a server or a terminal. An internal structure diagram of the computer device may be shown in FIG. 5. The computer device includes a processor, a memory, an input/output interface (I/O for short) and a communication interface. The processor, the memory, and the I/O interface are connected by a system bus. The communication interface is connected to the system bus by the I/O interface. The processor of the computer device is configured to provide computing and control capabilities. The memory of the computer device includes a non-volatile storage medium and an internal memory. The non-volatile storage medium stores an operating system, a computer program, and a database. The internal memory provides an environment for operation of an operating system and a computer program in the non-volatile storage medium. The I/O interface of the computer device is configured to exchange information between the processor and an external device. The communication interface of the computer device is configured to communicate with an external terminal through a network connection. When executed by a processor, the computer program implements the multimodal federated learning method based on a medical large model.

A person skilled in the art understands that the structure shown in FIG. 5 is a block diagram of just a part of the structure related to the solution of the present disclosure, and does not constitute any limitation on the computer device to which the solution of the present disclosure is applied. A specific computer device may include more or fewer components than those shown in the drawings, or may include a combination of some of the components, or may arrange the components in a different way. In an exemplary embodiment, a computer device is provided, including a memory and a processor. The memory stores a computer program. When executing the computer program, the processor implements the steps in each method embodiment described above.

In an exemplary embodiment, a computer-readable storage medium is provided. The computer-readable storage medium stores a computer program. When executed by a processor, the computer program implements the steps in each method embodiment described above.

In an exemplary embodiment, a computer program product is provided. The computer program product includes a computer program. When executed by a processor, the computer program implements the steps in each method embodiment described above.

It is hereby noted that the user information (including, but not limited to, user device information, personal information of the user) and data (including, but not limited to, data for analysis, stored data, displayed data) related to the present disclosure are information and data authorized by the user or duly authorized by all parties, and the acquisition, use, and processing of relevant data are required to comply with relevant rules and regulations.

A person of ordinary skill in the art may understand that all or some of the processes of the methods in the foregoing embodiments may be implemented by a computer program instructing relevant hardware. The computer program may be stored in a nonvolatile computer-readable storage medium. When executed, the computer program can perform processes that include the foregoing method embodiments. Any reference to a memory, a database, or another medium used in each embodiment of the present disclosure may include at least one of a non-volatile memory or a volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, or the like. Volatile memories may include a random-access memory (RAM), an external cache, or the like. Illustratively rather than restrictively, the RAM is in diverse forms, such as a static random access memory (SRAM) or a dynamic random access memory (DRAM).

The database mentioned in each embodiment of the present disclosure may include at least one of a relational database or a non-relational database. The non-relational database may include, but is not limited to, a blockchain-based distributed database. The processor mentioned in each embodiment of the present disclosure may be, but is not limited to, a general-purpose processor, central processing unit, graphics processing unit, digital signal processor, programmable logic device, quantum computing-based data processing logic device, or the like.

The technical features in the foregoing embodiments may be combined arbitrarily. For brevity, not all possible combinations of the technical features in the embodiments are described. However, to the extent that no conflict exists, all such combinations of the technical features are considered falling within the scope hereof.

Several examples are used herein for illustration of the principles and implementation methods of the present disclosure. The description of the above embodiments is intended to help understand the method and core principles of the present disclosure. To a person of ordinary skill in the art, various modifications and variations may be made to the specific implementation method and application scope of the present disclosure without departing from the essence of the present disclosure. In conclusion, the content of this specification is not to be construed as a limitation on the present disclosure.

What is claimed is:

1. A multimodal federated learning method based on a medical large model, wherein the multimodal federated learning method based on a medical large model comprises:
sending, by a server, an initial global model to each medical institution client, wherein the initial global model is a medical large model;
performing local training on the received initial global model on any one of the medical institution clients by use of a multimodal feature fusion dataset to obtain a local model, and uploading the local model to the server;
performing cross-validation on a plurality of local models on the server, and implementing global training by evaluating and retaining high-quality model features to obtain an optimized global model;
updating the initial global model to the optimized global model when the optimized global model fails to meet a first preset condition, and then returning to the step of sending the initial global model to each medical institution client through the server, so as to perform a next federated learning operation; iterating until the optimized global model meets the first preset condition whereupon the learning is stopped, marking the optimized global model as an optimal global model, and marking a local model corresponding to each medical institution client as an optimal local model;
clustering the medical institution clients based on the optimal global model to obtain a plurality of clusters, and then inputting the optimal local model of each medical institution client into the corresponding cluster to undergo intra-cluster aggregation to obtain a cluster model;
feeding the cluster model of each cluster into each corresponding medical institution client to undergo local training until a second preset condition is met, whereupon a secondary optimized local model is obtained; and
uploading the secondary optimized local model of each medical institution client to the server to undergo aggregation to obtain an optimal federated global model.

2. The multimodal federated learning method based on a medical large model according to claim 1, wherein a process of acquiring the multimodal feature fusion dataset comprises:
acquiring text data, voice data, and image data from the medical institution server;
extracting features of the text data, voice data, and image data of the medical institution server separately to obtain text features, voice features, and image features;
performing, based on an attention mechanism, multimodal feature fusion on the text features, the voice features, and the image features to obtain a multimodal feature fusion dataset.

3. The multimodal federated learning method based on a medical large model according to claim 1, wherein, in a process of performing local training on the received initial global model by use of a multimodal feature fusion dataset, the following loss function is used:
$${L}_{i} ( {f}_{i}^{p} ) = \frac{1}{{D}_{i}} {\sum }_{k ∈ {D}_{i}} {F}_{k} ( {f}_{i}^{p} )$$
wherein,
$${L}_{i} ( {f}_{i}^{p} )$$
is a value of the loss function of
$${f}_{i}^{p} , {f}_{i}^{p}$$
is a model feature of a medical institution client i in a p^{th} round, Dᵢ is multimodal feature fusion dataset of the medical institution client i, k is any one multimodal feature fusion datum in Dᵢ, and a purpose of
$${F}_{k} ( {f}_{i}^{p} )$$
is to find an optimal parameter ƒ* of the model to minimize the loss function; and ƒ* is formalized as:
$${f}^{*} = arg min {L}_{i} ( {f}_{i}^{p} ) ;$$
and
updating
$$F ( {f}_{i}^{p} )$$
iteratively for several times by a gradient descent method to minimize a local loss by using the following formula:
$${f}_{i}^{p} = {f}_{i}^{p - 1} - λ ∇ F ( {f}_{i}^{p} ) ,$$
wherein, λ is a step length,
$${f}_{i}^{p - 1}$$
is a model feature of the medical institution client i in a (p−1)^{th} round, and
$$∇ F ( {f}_{i}^{p} )$$
is a gradient operator.

4. The multimodal federated learning method based on a medical large model according to claim 1, wherein a function formula of the optimized the global model is:
$${f}^{p + 1} = \frac{{\sum }_{i = 1}^{N} ω \vert {D}_{i} \vert {f}_{i}^{p}}{{\sum }_{i = 1}^{N} \vert {D}_{i} \vert}$$
wherein, ƒ^{p+1} is the optimized global model in a (p+1)^{th} round, N is a number of medical institution clients; ω is a model feature weight;
$${f}_{i}^{p}$$
is a model feature of the medical institution client i in the p^{th} round, and Dᵢ is the multimodal feature fusion dataset of the medical institution client i.

5. The multimodal federated learning method based on a medical large model according to claim 1, wherein the clustering the medical institution clients based on the optimal global model to obtain a plurality of clusters comprises:
sending, by the server, the optimal global model to each medical institution client, performing local training to obtain an optimized local model, and uploading the optimized local model to the server;
performing feature mapping on the plurality of the optimized local models by using a multi-layer neural network, so as to obtain a feature mapping graph, wherein the model feature of each optimized local model is converted into a weight of each edge in the corresponding feature mapping graph;
performing personalized enhanced feature extraction on the feature mapping graph to obtain a feature enhancement graph;
converting the feature enhancement graph into a similarity distance vector;
clustering the plurality of medical institution clients by using a clustering algorithm based on the similarity distance vector, so as to obtain a plurality of clusters.

6. The multimodal federated learning method based on a medical large model according to claim 5, wherein the converting the feature enhancement graph into a similarity distance vector comprises:
generating a weighted adjacency matrix based on the feature enhancement graph;
obtaining a Laplacian operator of the feature enhancement graph based on an adjoint diagonal matrix of the weighted adjacency matrix;
calculating an eigenvalue sequence and an eigenvector sequence of the Laplacian operator of the feature enhancement graph;
calculating eigenvalue similarity based on the eigenvalue sequence by using the following formula:
$${Dis}_{eig} ( i , j ) = \sqrt{{\sum }_{k = 1}^{n} {( {λ}_{ik} - {λ}_{jk} )}^{2}} ,$$
calculating eigenvector similarity based on the eigenvector sequence by using the following formula:
$$D i {s}_{v e c} ( i , j ) = \sqrt{{\sum }_{k = 1}^{n} {\sum }_{I = 1}^{n} {( {v}_{i , kI} - {v}_{j , kI} )}^{2}} ,$$
performing weighting by using the following formula, so as to calculate global similarity:
$$D i {s}_{c o m b} ( i , j ) = α {Dis}_{e i g} ( i , j ) + ( 1 - α ) {Dis}_{v e c} ( i , j ) ,$$
storing the global similarity of each feature enhancement graph as the similarity distance vector of the corresponding medical institution client,
wherein, α∈[0,1] is a weight parameter, Dis_{comb} (i, j) is the global similarity between the feature enhancement graph
$${G}_{i}^{′}$$
and the feature enhancement graph
$${G}_{j}^{′} ;$$
Dis_{eig}(i, j) is the eigenvalue similarity between the feature enhancement graph
$${G}_{i}^{′}$$
and the feature enhancement graph
$${G}_{j}^{′} ;$$
Dis_{vec}(i, j) is the eigenvector similarity between the feature enhancement graph
$${G}_{i}^{′}$$
and the feature enhancement graph
$${G}_{j}^{′} ;$$
λᵢₖ is a k^{th} eigenvalue in the feature enhancement graph
$${G}_{i}^{′} ;$$
λⱼₖ is a k^{th} eigenvalue in the feature enhancement graph
$${G}_{j}^{′} ;$$
v_{i,kl} is a kl^{th} eigenvector in the feature enhancement graph
$${G}_{i}^{′} ;$$
and v_{j,kl} is a kl^{th} eigenvector in the feature enhancement graph
$${G}_{j}^{′} .$$

7. The multimodal federated learning method based on a medical large model according to claim 5, wherein a function used in the step of performing personalized enhanced feature extraction on the feature mapping graph is:
$${H}_{t}^{l} = \{ \begin{matrix}{1 , \vert ω ( {e}_{t}^{l} ) - ω ) {e}_{t}^{l} )}_{i} \vert \geq δ , {e}_{t}^{l} ∈ {E}_{l}^{P} , {E}_{l , i}^{P + 1} \\ {0 , \vert ω ( {e}_{t}^{l} ) - ω ) {e}_{t}^{l} )}_{i} \vert < δ , {e}_{t}^{l} ∈ {E}_{l}^{P} , {E}_{l , i}^{P + 1}\end{matrix} .$$
wherein, when a value of
$${H}_{t}^{l}$$
is 1, the value means that the feature weight is valid before and after the feature mapping graph is enhanced, and an edge
$${e}_{t}^{l}$$
is retained; when the value of
$${H}_{t}^{l}$$
is 0, the value means that the feature weight is invalid before and after the feature mapping graph is enhanced, and the edge
$${e}_{t}^{l}$$
is deleted;
$$ω ( {e}_{t}^{l} ) and ω {( {e}_{t}^{l} )}_{i}$$
are weights of the edge
$${e}_{t}^{l}$$
before and after the feature mapping graph is enhanced, respectively; δ is a threshold; and
$${E}_{l}^{P} and {E}_{l , i}^{P + 1}$$
are a set of edges connecting an (s−1)^{th} layer and an s^{th} layer before and after the feature mapping graph is enhanced, respectively, satisfying: 1≤s≤S.

8. The multimodal federated learning method based on a medical large model according to claim 1, wherein the cluster model is:
$${f}_{j}^{P + 1} = \frac{{\sum }_{{n}_{j} = 1}^{{N}_{j}} \vert {D}_{{n}_{j}} \vert {f}_{{n}_{j}}^{P + 1}}{{\sum }_{{n}_{j} = 1}^{{N}_{j}} \vert {D}_{{n}_{j}} \vert} ,$$
wherein,
$${f}_{j}^{P + 1}$$
is the cluster model in a (P+1)^{th} round, Dₙ_{ⱼ} is the multimodal feature fusion dataset of a medical institution client nⱼ corresponding to a j^{th} cluster,
$${f}_{{n}_{j}}^{P + 1}$$
is the optimal local model of the medical institution client nⱼ corresponding to the j^{th} cluster in the (P+1)^{th} round, Nⱼ is a number of medical institution clients in the j^{th} cluster, satisfying:
$${\sum }_{j = 1}^{J} {N}_{j} = N ,$$
wherein N is the number of medical institution clients.

9. A multimodal federated learning system based on a medical large model, wherein the multimodal federated learning system based on a medical large model comprises:
a sending module, configured to send an initial global model to each medical institution client through a server, wherein the initial global model is a medical large model;
a local multi-round training module, configured to perform local training on the received initial global model on any one of the medical institution clients by use of a multimodal feature fusion dataset to obtain a local model, and upload the local model to the server;
a cross-validation optimum module, configured to perform cross-validation on a plurality of local models on the server, and implement global training by evaluating and retaining high-quality model features to obtain an optimized global model;
a cyclic learning module, configured to: update the initial global model to the optimized global model when the optimized global model fails to meet a first preset condition, and then return to the step of sending the initial global model to each medical institution client through the server, so as to perform a next federated learning operation; repeat this process until the optimized global model meets the first preset condition whereupon the learning is stopped, mark the optimized global model as an optimal global model, and mark a local model corresponding to each medical institution client as an optimal local model;
a clustering module, configured to cluster the medical institution clients based on the optimal global model to obtain a plurality of clusters, and then input the optimal local model of each medical institution client into the corresponding cluster to undergo intra-cluster aggregation to obtain a cluster model;
wherein the sending module is further configured to: feed the cluster model of each cluster into each corresponding medical institution client to undergo local training in the local multi-round training module until a second preset condition is met, whereupon a secondary optimized local model is obtained; and
an ultimate model determining module, configured to: aggregate the secondary optimized local model of each medical institution client uploaded to the server, so as to obtain an optimal federated global model.

10. A computer device, comprising: a memory, a processor, and a computer program stored in the memory and executable on the processor, wherein the processor executes the computer program to implement the multimodal federated learning method based on a medical large model according to claim 1.

11. The computer device according to claim 10, wherein a process of acquiring the multimodal feature fusion dataset comprises:
acquiring text data, voice data, and image data from the medical institution server;
extracting features of the text data, voice data, and image data of the medical institution server separately to obtain text features, voice features, and image features;
performing, based on an attention mechanism, multimodal feature fusion on the text features, the voice features, and the image features to obtain a multimodal feature fusion dataset.

12. The computer device according to claim 10, wherein, in a process of performing local training on the received initial global model by use of a multimodal feature fusion dataset, the following loss function is used:
$${L}_{i} ( {f}_{i}^{p} ) = \frac{1}{{D}_{i}} {\sum }_{k ∈ {D}_{i}} {F}_{k} ( {f}_{i}^{p} )$$
wherein,
$${L}_{i} ( {f}_{i}^{p} )$$
is a value of the loss function of
$${f}_{i}^{p} , {f}_{i}^{p}$$
is a model feature of a medical institution client i in a p^{th} round, Dᵢ is multimodal feature fusion dataset of the medical institution client i, k is any one multimodal feature fusion datum in Dᵢ, and a purpose of
$${F}_{k} ( {f}_{i}^{p} )$$
is to find and optimal parameter ƒ* of the model to minimize the loss function; and ƒ* is formalized as:
$${f}^{*} = arg min {L}_{i} ( {f}_{i}^{p} ) ;$$
and
updating
$$F ( {f}_{i}^{p} )$$
iteratively for several times by a gradient descent method to minimize a local loss by using the following formula:
$${f}_{i}^{p} = {f}_{i}^{p - 1} - λ ∇ F ( {f}_{i}^{p} ) ,$$
wherein, λ is a step length,
$${f}_{i}^{p - 1}$$
is a model feature of the medical institution client i in a (p−1)^{th} round, and
$$∇ F ( {f}_{i}^{p} )$$
is a gradient operator.

13. The computer device according to claim 10, wherein a function formula of the optimized the global model is:
$${f}^{p + 1} = \frac{{\sum }_{i = 1}^{N} ω \vert {D}_{i} \vert {f}_{i}^{p}}{{\sum }_{i = 1}^{N} \vert {D}_{i} \vert}$$
wherein, ƒ^{p+1} is the optimized global model in a (p+1)^{th}-round, N is a number of medical institution clients; ω is a model feature weight;
$${f}_{i}^{p}$$
is a model feature of the medical institution client i in the p^{th} round, and is the multimodal feature fusion dataset of the medical institution client i.

14. The computer device according to claim 10, wherein the clustering the medical institution clients based on the optimal global model to obtain a plurality of clusters comprises:
sending, by the server, the optimal global model to each medical institution client, performing local training to obtain an optimized local model, and uploading the optimized local model to the server;
performing feature mapping on the plurality of the optimized local models by using a multi-layer neural network, so as to obtain a feature mapping graph, wherein the model feature of each optimized local model is converted into a weight of each edge in the corresponding feature mapping graph;
performing personalized enhanced feature extraction on the feature mapping graph to obtain a feature enhancement graph;
converting the feature enhancement graph into a similarity distance vector;
clustering the plurality of medical institution clients by using a clustering algorithm based on the similarity distance vector, so as to obtain a plurality of clusters.

15. The computer device according to claim 14, wherein the converting the feature enhancement graph into a similarity distance vector comprises:
generating a weighted adjacency matrix based on the feature enhancement graph;
obtaining a Laplacian operator of the feature enhancement graph based on an adjoint diagonal matrix of the weighted adjacency matrix;
calculating an eigenvalue sequence and an eigenvector sequence of the Laplacian operator of the feature enhancement graph;
calculating eigenvalue similarity based on the eigenvalue sequence by using the following formula:
$$D i {s}_{eig} ( i , j ) = \sqrt{{\sum }_{k = 1}^{n} {( {λ}_{ik} - {λ}_{jk} )}^{2}} ,$$
calculating eigenvector similarity based on the eigenvector sequence by using the following formula:
$$D i {s}_{v e c} ( i , j ) = \sqrt{{\sum }_{k = 1}^{n} {\sum }_{I = 1}^{n} {( {v}_{i , kl} - {v}_{j , kl} )}^{2}} ,$$
performing weighting by using the following formula, so as to calculate global similarity:
$${Dis}_{c o m b} ( i , j ) = α {Dis}_{e i g} ( i , j ) + ( 1 - α ) {Dis}_{v e c} ( i , j ) ,$$
storing the global similarity of each feature enhancement graph as the similarity distance vector of the corresponding medical institution client,
wherein, α∈[0,1] is a weight parameter, Dis_{comb} (i, j) is the global similarity between the feature enhancement graph
$${G}_{i}^{′}$$
and the feature enhancement graph
$${G}_{j}^{′} ;$$
Dis_{eig}(i, j) is the eigenvector similarity between the feature enhancement graph
$${G}_{i}^{′}$$
and the freatrue enhancement graph
$${G}_{j}^{′} ;$$
Dis_{vec}(i, j) is the eigen vector similarity between the feature enhancement graph
$${G}_{i}^{′}$$
and the feature enhancement graph
$${G}_{j}^{′} ;$$
λᵢₖ is a k^{th} eigenvalue in the feature enhancement graph
$${G}_{i}^{′} ;$$
λⱼₖ is a k^{th} eigen value in the feature enhancement graph
$${G}_{j}^{′} ;$$
v_{i,kl} is a kl^{th} eigenvector in the feature enhancement graph
$${G}_{i}^{′} ;$$
and v_{j,kl} is a kl^{th} eigenvector in the feature enhancement graph
$${G}_{i}^{′} ;$$

16. The computer device according to claim 14, wherein a function used in the step of performing personalized enhanced feature extraction on the feature mapping graph is:
$${H}_{t}^{l} = \{ \begin{matrix}1 , \vert ω ( {e}_{t}^{l} ) - {ω ( {e}_{t}^{l} )}_{i} \vert \geq δ , {e}_{t}^{l} ∈ {E}_{l}^{P} , {E}_{l , i}^{P + 1} \\ 0 , \vert ω ( {e}_{t}^{l} ) - {ω ( {e}_{t}^{l} )}_{i} \vert < δ , {e}_{t}^{l} ∈ {E}_{l}^{P} , {E}_{l , i}^{P + 1}\end{matrix} ,$$
wherein, when a value of
$${H}_{t}^{l}$$
is 1, the value means that the feature weight is valid before and after the feature mapping graph is enhanced, and an edge
$${e}_{t}^{l}$$
is retained; when the value of
$${H}_{t}^{l}$$
is 0, the value means that the feature weight is invalid before and after the feature mapping graph is enhanced, and the edge
$${e}_{t}^{l}$$
is deleted;
$$ω ( {e}_{t}^{l} ) and {ω ( {e}_{t}^{l} )}_{i}$$
are weights of the edge
$${e}_{t}^{l}$$
before and after the feature mapping graph is enhanced, respectively; δ is a threshold; and
$${E}_{l}^{P} and {E}_{l , i}^{P + 1}$$
are a set of edges connecting an (s−1)^{th} layer and an s^{th} layer before and after the feature mapping graph is enhanced, respectively, satisfying: 1≤s≤S.

17. The computer device according to claim 10, wherein the cluster model is:
$${f}_{j}^{P + 1} = \frac{{\sum }_{{n}_{j} = 1}^{{N}_{j}} \vert {D}_{{n}_{j}} \vert {f}_{{n}_{j}}^{P + 1}}{{\sum }_{{n}_{j} = 1}^{{N}_{j}} \vert {D}_{{n}_{j}} \vert} ,$$
wherein,
$${f}_{j}^{P + 1}$$
is the cluster model in a (P+1)^{th} round, Dₙ_{ⱼ} is the multimodal feature fusion dataset of a medical institution client nⱼ corresponding to a j^{th} cluster,
$${f}_{{n}_{j}}^{P + 1}$$
is the optimal local model of the medical institution client nⱼ corresponding to the j^{th} cluster in the (P+1)^{th} round, Nⱼ is a number of medical institution clients in the j^{th} cluster, satisfying:
$${\sum }_{j = 1}^{J} {N}_{j} = N ,$$
wherein N is the number of medical institution clients.
